(19) United States
(12) Patent Application Publication
(10) Pub. No.: US 2018/0129029 A1
(43) Pub. Date: May 10, 2018

(54) DEBRIS DIRECT REFLECTION EMBEDDED MICROSCOPE FOR ON-LINE VISUAL FERROGRAPH

(71) Applicant: Xi'an Jiaotong University, Xi'an (CN)

(72) Inventors: Junhong MAO, Xi'an (CN); Bo LI, Xi'an (CN); Bin FAN, Xi'an (CN); Donghui ZHENG, Xi'an (CN); You-Bai XIE, Xi'an (CN)

(21) Appl. No.: 15/806,232

(22) Filed: Nov. 7, 2017

(30) Foreign Application Priority Data
Nov. 7, 2016 (CN) 201610976289.X

(51) Int. Cl. G02B 21/02; G01N 33/28; G01N 15/06; G02B 21/00; G02B 21/06; G02B 21/36; G06T 7/00

(52) U.S. Cl. CPC G02B 21/025; G01N 33/2835; G01N 15/06; G02B 21/0016; G06T 2207/10152; G02B 21/365; G06T 7/0004; G01N 2015/0693; G02B 21/06

(57) ABSTRACT

A debris direct reflection embedded microscope comprises a focus mechanism 1 and a reflected light source 5 into the pole gap of the electromagnetic debris deposition device 3 for on-line visual ferrograph. A reflection ferrogram is imaged directly other than though the oil film in the oil tunnel. The focus mechanism 1 is composed of a lens sleeve 10, a limit threaded sleeve 11, a focus threaded sleeve 14 and a connecting seat 2. An image sensor 13 is on the top of the focus mechanism 1 and a lens 12 is inside the lens sleeve 10. The focus mechanism 1 and reflected light source 5 are placed in the internal space of the electromagnetic debris deposition device 3, and the center optical axis of the focus mechanism 1 coincides with the center axis of the electromagnetic debris deposition device 3. The focus mechanism 1 and reflected light source 5 are fixed in the connecting seat 2, and the connecting seat 2 connects the focus mechanism 1 and the magnetic poles 3-1 together. A transmission light source 6 is a bi-color flat light source. Resolution and image contrast of ferrograms are improved by adjusting intensities of the reflected light source 5 and transmission light source 6.

### FIELD OF THE INVENTION

This invention concerns a digital microscopic imaging apparatus and method for on-line wear debris monitoring. In particular, embodiments of the present invention concern a debris direct reflection embedded microscope for on-line visual ferrograph.

### BACKGROUND OF THE INVENTION

The information of wear debris in the lubricating oil can reflect the wear condition of mechanical equipment. According to the concentration and characteristics of wear debris, the wear degree, operate conditions and wear trends of the mechanical equipment can be monitored.

The existing wear monitoring technology can be used to obtain the wear information of the mechanical equipment. Non-visual monitoring is one of the wear monitoring methods, U.S. Pat. No. 8,522,604B2, herein incorporated by reference, discloses a metal wear detection apparatus and method for detection of wear particles in a lubricant, this detection system in coupled with the electrodes for detection of wear particles passing through the micro-channel, based on a change in capacitance of the electrodes. U.S. Pat. No. 7,172,903 B2, herein incorporated by reference, discloses a method for on-line monitoring oil aging and particulate buildup in a lubricant by detecting light transmitted through the lubricant at a wavelength preselected from a specific range of wavelengths. Although these methods can reflect the wear trends of the mechanical equipment, the characteristic information of wear debris cannot be detected by non-visual monitoring.

In order to obtain the characteristics of wear debris, it is necessary to obtain the image of wear debris. There are two kinds of visual wear monitoring methods, one is off-line visual ferrograph, and the other is on-line visual ferrograph. The commonly used off-line visual ferrograph is that of analytical ferrograph, U.S. Pat. No. 4,047,814, herein incorporated by reference, discloses a method and apparatus for segregating particulate matter, this approach was the first describe of deposit wear particles by passing the liquid through a magnetic field. The analytical ferrograph is based on this method, wear debris are separated by magnetic force and made into ferrogram, and the characteristics of wear debris can be observed by an optical microscope. However, the size of analytical ferrograph device is large, and it cannot be used for real-time analysis because of the complex process.

In recent years, the on-line visual ferrograph technology has developed rapidly. Because of the high reliability of wear data, it can identify abnormal wear and provide early warnings for failure prevention. Most existing on-line visual monitoring devices use transmission light as the light source to obtain wear debris images. U.S. Pat. No. 7,385,694B2, herein incorporated by reference, discloses an image analysis detector for gathering images of the oil, which includes an imaging device for detecting debris in the fluid illuminated by laser, and generating object segments representative of the debris and sending the object segments to the general purpose computer for analysis. U.S. Pat. No. 9,341,612B2, herein incorporated by reference, discloses a system and method for monitoring a fluid, this kind of system has a lens situated between the image capture system and the oil flow, and the lighting system comprises a polarisation control system of at least one LED diode configured to avoid emission fluctuations due to changes in temperature. Other countries also have on-line visual ferrograph patents, for example, CN. Pat. No. 200610041773.X, herein incorporated by reference, discloses a small on-line visual ferrograph detector for collecting the image information of wear debris. This detector contains an electromagnetic debris deposition device, which is used to realize the ordered deposition of wear debris in the collected area. The indexes of particle coverage area (IPCA) are used to reflect wear debris concentration. However, in practice, the transmittance of oil has a great influence on the on-line wear monitoring. When the transmission light is used to detect wear debris information, the transmission light need to pass through the oil layer, but the bubbles and impurities in the oil impede the resolution of wear debris imaging and wear debris feature extraction. Additionally, when the transmittance of the oil is low, the deposited wear debris cannot be clearly imaged under transmission light.

U.S. Pat. No. 9,274,041, herein incorporated by reference, discloses a particle counter and classification system including an imaging subsystem configured to determine the size and morphology of particles above a predetermined size in a fluid in a sample cell. A detector is a component of the imaging subsystem including a light source directing electromagnetic radiation into the sample cell and the detector is responsive to electromagnetic radiation emitted from the sample cell. In the system, the information of wear debris in the low transmittance oil can be obtained by using the reflected light, but there are still some problems. One problem is that the quality of a reflected ferrogram is too low to be used. The main reason is non-uniform lamination in image plane. Another problem is that if monochromatic light is used for lighting, when the color of wear debris and oil is similar or the oil under light-proof condition, the imaging device will lose wear debris image acquisition function. These problems seriously affected the accuracy and reliability of the wear monitoring results.

### SUMMARY OF THE INVENTION

According to the present invention, a debris direct reflection embedded microscope for on-line visual ferrograph is provided. The present invention incorporates a focus mechanism, a reflected light source and a transmission light source. The focus mechanism is composed of a focus threaded sleeve, a limit threaded sleeve, a lens sleeve and a connecting seat. An image sensor is mounted on the top of the focus mechanism and a lens is fixed inside the lens sleeve. Different magnifications of the debris imaging can be achieved by adjusting the position of the limit threaded sleeve, and the focusing of image sensor can be achieved by adjusting the focus threaded sleeve.

The focus mechanism and the reflected light source are installed in the internal space of the electromagnetic debris deposition device, and the center optical axis of the focus mechanism coincides with the center axis of the electromagnetic debris deposition device. The focus mechanism and reflected light source are fixed in the connecting seat, and the connecting seat connects the focus mechanism and the magnetic poles together. This structure arrangement realized the miniaturization and achieved micro-imaging of debris under short optical structure.

A transmission light source is a bi-color flat light source with different wavelengths. Quasi-uniform illumination can be achieved by using the bi-color flat light source as the background light source. The color selection of the transmission light source is based on the transmittance of the oil. In term of adjusting the light intensity of the reflected light source and the transmission light source, wear debris can be monitored in low transmittance oil. Thus, the quality of reflected ferrogram and the resolution and the image contrast of ferrograms are improved.

### BRIEF DESCRIPTION OF THE DRAWINGS

The present invention description below refers to the accompanying drawings, in which identical reference numerals refer to the same component, of which:

FIG. 1 is a schematic diagram of the structure of the present invention;

FIG. 2 is a schematic diagram of the structure of a focus mechanism;

FIG. 3 is a schematic diagram of structure of a transmission light resource;

FIG. 4 is a flow chart of an adjustment method of the light intensity of transmission light source and reflected light source;

FIG. 5A is a ferrogram of the gearbox oil detected by the present invention;

FIG. 5B is a ferrogram of the gearbox oil detected by the existing technology;

FIG. 5C is a ferrogram of the gasoline engine oil detected by the present invention;

FIG. 5D is a ferrogram of the gasoline engine oil detected by the existing technology;

FIG. 5E is a ferrogram of the diesel engine oil detected by the present invention;

FIG. 5F is a ferrogram of the diesel engine oil detected by the existing technology.

### DETAILED DESCRIPTION OF THE INVENTION

FIG. 1 is a schematic diagram of the structure of the present invention. A focus mechanism 1 is assembled with an electromagnetic debris deposition device 3 and an oil tunnel 4. The electromagnetic debris deposition device 3 is composed of two magnetic poles 3-1, two electromagnetic coils 3-2 and a U-type magnetic core 3-3. The oil tunnel 4 is fixed directly below the magnetic poles 3-1. The focus mechanism 1 and the reflected light source 5 are fixed to one side of the oil tunnel 4, and a transmission light source 6 is mounted on the other side of the oil tunnel 4. The focus mechanism 1 and reflected light source 5 are placed in the internal space of the electromagnetic debris deposition device 3 between two electromagnetic coils 3-2, and the symmetry center of two magnetic poles 3-1 coincide with the central axis of the electromagnetic debris deposition device 3. Similarly, the center optical axis of the focus mechanism 1 coincides with the center axis of the electromagnetic debris deposition device 3. This kind of structure is tighter and the size is small.

The focus mechanism 1 comprises a lens sleeve 10, a limit threaded sleeve 11, a focus threaded sleeve 14 and a connecting seat 2. An image sensor 13 is on the top of the focus mechanism 1 and a lens 12 is inside the lens sleeve 10. The focus mechanism 1 is fixedly connected to the connecting seat 2 by the set screws 9 on the both sides of the limit threaded sleeve 11, and the connecting seat 2 connects the focus mechanism 1 and the magnetic poles 3-1 together. The reflected light source 5 is fixedly connected to the lower end of the lens sleeve 10 by screws, and the reflected light source 5 and the lens sleeve 10 are tightly fixed together. In addition, thermal silica gel is uniform coated on the bonding surface. The connecting seat 2 is fixedly connected with the oil tunnel 4, and two magnetic poles 3-1 are sandwiched between these two parts so that it can realize the optical path sealing of the reflected light imaging device and eliminate the influence of the stray light interference on the quality of ferrograms. Two electromagnetic coils 3-2 wound round the U-type magnetic core 3-3, and two magnetic poles 3-1 (N and S) are fixedly connected to the U-type magnetic core 3-3 and the oil tunnel 4 by screws.

A transmission light source 6 is fixed in the screw hole of the oil tunnel 4, and an optical lens 7 is fixed directly above the transmission light source 6 for correcting the direction of the transmission light. An oil chamber 8 is above the optical lens 7, and the transmission light passes through a bottom optical glass window 8-1 of the oil chamber 8 to achieve uniform illumination of the transmission light. Additionally, in order to ensure that the high-gradient magnetic field generated by the magnetic poles 3-1 can effectively acquire the debris in the oil, the upper surface of a top optical glass window 8-2 is closely fitted with two magnetic poles 3-1.

The debris in the oil are orderly deposited on the lower surface of the top optical glass window 8-2 by the high gradient magnetic field of the magnetic poles 3-1, so that the reflected light can image without passing through the oil. Compared with the existing online image visual ferrography technology, the present invention can avoid the influence of the low transmittance oil and impurities in the oil and it also can improve the image resolution of ferrograms. In addition, the bottom optical glass window 8-1 and the top optical glass window 8-2 of the oil chamber 8 are optical glasses having a transmittance of more than 95%.

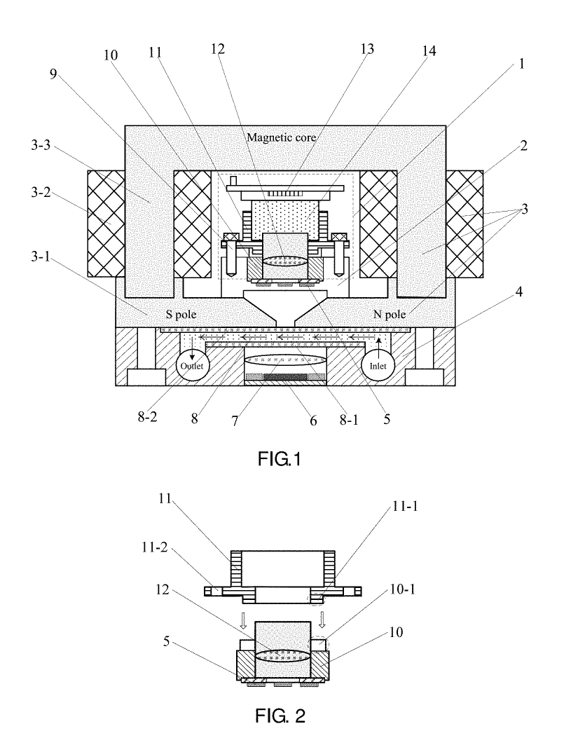

In FIG. 1 and FIG. 2, there is an internal thread in the lens sleeve 10 of the focus mechanism 1, the lens 12 is fitted into the lens sleeve 10 by the internal thread, and the combination of the lens sleeve 10 and the lens 12 is mounted in the connecting seat 2 by an external thread at the lower end of the lens sleeve 10. The upper end of the lens sleeve 10 has several slots 10-1, and the lower end of the limit threaded sleeve 11 has the same amount of bosses 11-1. Therefore, the limit threaded sleeve 11 can be mounted in the slots 10-1 by the bosses 11-1, and different magnifications of the debris imaging can be achieved by adjusting the position of the limit threaded sleeve 11. Furthermore, there are two through holes 11-2 at the both side of the limit threaded sleeve 11, the focus mechanism 1 and the connecting seat 2 can be connected together by screwing the set screws 9 into the through holes 11-2 when after adjustment of object distance. Moreover, the lower end of the focus threaded sleeve 14 is connected with the limit threaded sleeve 11 by external thread of the outer side of the focus threaded sleeve 14, and the upper end of the focus threaded sleeve 14 is fixedly connected with image sensor 13. The focusing of image sensor 13 can be achieved by adjusting the focus threaded sleeve 14.

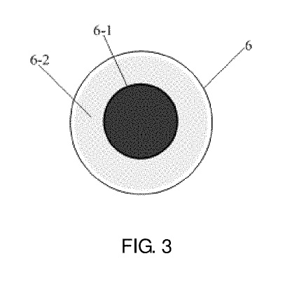

FIG. 3 shows that the transmission light source 6 is a bi-color flat light source composed of two monochromatic light A and B, a light source A 6-1 is installed in the center, and a light source B 6-2 is arranged at the edge of the light source A 6-1. The light source B 6-2 can achieve quasi-uniform illumination through illumination superposition. Operating at the same driving current, the light intensity of the light source A 6-1 is equal to the light source B 6-2. Experimental results indicate that the longer the wavelength of light, the less light energy loss of oil. In order to reduce the loss of light energy and improve the transmittance of the oil, in an embodiment the light source A 6-1 is set to red light and the light source B 6-2 is set to yellow. The choice of light source color is according to the transmittance of the oil. In the embodiment the light source B 6-2 is used as transmission light source when the transmittance of the oil is more than 50%, and the light source A 6-1 is used as the transmission light source when the transmittance of the oil is less than or equal to 50%. The purpose is to reduce the influence of light absorption and light scattering of oil on the image quality of wear debris.

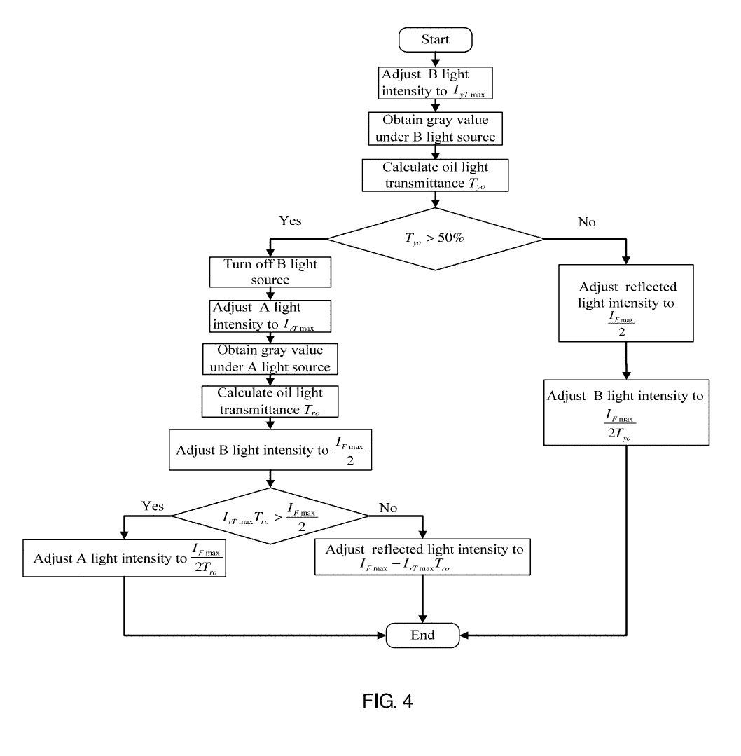

An adjustment method of the light intensity of transmission light source and reflected light source is given in FIG. 4. According to the different transmittances of the oil, the light intensity of the light source A 6-1 or the light source B 6-2 are adjusted to equal with the intensity of the reflected light in the oil chamber 8, so that the debris image can be reliable acquired, and the resolution and the image contrast of ferrograms can be improved. To ensure the sharpness of the debris imaging is not affected under non-oiling condition, and to improve the reliability of on-line visual ferrograph for oil monitoring. The light source A 6-1 or the light source B 6-2 is used as the transmission light source 6, so the light energy loss in the oil can be reduced, especially for on-line monitoring in low transmittance oil. Using reflected light for imaging can improve the image resolution, moreover, using the light source A or the light source B as the image background color can ensure that the color deviation and image contrast between the background and debris is large enough to reduce the difficulty of debris visual feature information extraction.

To control the transmission light and reflected light intensity, firstly on-line ferrograph imaging device linear working range in the oil chamber 8 under non-oiling condition is measured, then the relationship between the average gray value of transmission light image and the light intensity of deposition of oil chamber 8 can be obtained by

$$2 {\sum }_{i = M / 4}^{3 M / 4} {\sum }_{j = 1}^{N} G ( {x}_{i} , {y}_{j} ) / MN = {aI}_{T} {T}_{gls} + b$$

where M, N are the rows and columns of the image sensor 13 pixel array respectively; M×N is the size of the image sensor 13; G(xᵢ,yⱼ) is the pixel gray value of the image at (xᵢ,yⱼ); a is the ratio of the light intensity to the gray value of the image sensor 13, and b is the constant determined by the sensitivity of the image sensor 13; I_{T} is the light intensity of transmission light source 6; T_{gls} is the transmittance of the optical glasses used in oil tunnel 4; I_{T}T_{gls} is the light intensity of deposition of oil chamber 8. Additionally, by using the light source A 6-1 and the light source B 6-2 as the transmission light source 6 respectively, different transmission light intensities corresponding average grey value of image are measured, and the relationship between the average gray value of the transmission light image and the light intensity of deposition of oil chamber 8 is obtained by linear fitting method.

In the normal operating range of the light source, the light intensity is proportional to the magnitude of the driving current. The corresponding relationship between the driving current and the light intensity of the light source A 6-1, light source B 6-2 and the reflected light source 5 is obtained, and the light intensity of the transmission light source 6 and reflected light source 5 can be adjusted by changing the current according to this corresponding relationship.

In the embodiments the maximum light intensity of light source A 6-1 is I_{A max}; the maximum light intensity of light source B 6-2 is I_{B max}, and the maximum light intensity of reflected light source 5 is I_{F max}. The steps of work flow and light intensity adjustment of on-line visual ferrograph method are shown as follows:

Step one, using the image sensor 13 to obtain the background image when the light intensity of light source B 6-2 is the maximum I_{B max}; by extracting the average gray value from the illuminated area of the image the transmittance of the oil T_{B} can be calculated. Thus the transmittance of the oil T_{B} is given by

$${T}_{B} = \frac{2 {\sum }_{i = M / 4}^{3 M / 4} {\sum }_{j = 1}^{N} {G}_{Bmax} ( {x}_{i} , {y}_{j} ) / MN - {b}_{B}}{{a}_{B} {I}_{Bmax}} \times 100 \%$$

where G_{Bmax}(xᵢ,yⱼ) is the pixel gray value of the background image at (xᵢ,yⱼ); b_{B} is the constant of the sensitivities of the image sensor 13 under light B; a_{B} is the ratio of light intensity of the image sensor 13 to the gray value under light B.

Step two, to determine the size of T_{B}; the color of transmission light is selected and the light intensity of transmission light is adjusted.

If T_{B} is greater than 50%, the light source B 6-2 as the background light source is used and the reflected light source 5 is switched on, the light intensity of reflected light source 5 is set to one-half of the maximum I_{Fmax}; in order to ensure the light intensity of transmission light and reflected light on the top optical glass window 8-2 is the same, the light intensity of light source B 6-2 is set as I_{B}=I_{F max}/2T_{B}.

However, if T_{B} is less than or equal to 50%, the light source B 6-2 is switched off and the light source A 6-1 is switched on, the light intensity of light source A 6-1 is set to the maximum I_{A max}, then the image sensor 13 is used to obtain the background image, by extracting the average gray value from the illuminated area of the image the transmittance of the oil T_{A} can be calculated. Thus the transmittance of the oil T_{A} is given by

$${T}_{A} = \frac{2 {\sum }_{i = M / 4}^{3 M / 4} {\sum }_{j = 1}^{N} {G}_{Amax} ( {x}_{i} , {y}_{j} ) / MN - {b}_{A}}{{a}_{A} {I}_{Amax}} \times 100 \%$$

where G_{Amax}(xᵢ,yⱼ) is the pixel gray value of the background image at (xᵢ,yⱼ); b_{A} is the constant of the sensitivities of the image sensor 13 under light A; a_{A} is the ratio of light intensity of the image sensor 13 to the gray value under light A.

Then the light source A 6-1 is used as the background light source and the light intensity of reflected light source 5 is set to I_{F max}/2. When I_{Amax}T_{A}>I_{F max}/2, in order to ensure the light intensity of transmission light and reflected light on the top optical glass window 8-2 is the same, the light intensity of light source A 6-1 is set to I_{A}=I_{F max}/2T_{A}; when I_{Amax}T_{A}≤F max/2, the light source A 6-1 maintains the maximum light intensity, the difference between transmission light source 6 is used as the dimming reference, and the light intensity I_{F} of reflected light source 5 is set as I_{F}=I_{Fmax}−I_{A max}T_{A}.

In the embodiments, the light intensity of transmission light source 6 and reflected light source 5 are adjusted through the described method to ensure the light intensity between the background and debris is large enough to improve the image contrast and reduce the difficulty of debris visual feature information extraction. Adjustment of the light intensity of transmission light source 6 and reflected light source 5 can reduce the energy loss of the transmission light in the oil, and high quality images of wear debris from the low transmittance oil can be obtained, which solves the poor quality of wear debris imaging problem that caused by bubbles and impurities in the oil.

Finally, set up the deposition parameters to deposit wear particles orderly. The magnetomotive force of the electromagnetic debris deposition device 3 is arranged at 3001200 ampere turns and the flow rate of oil is varied from 2 mL/min to 10 mL/min.

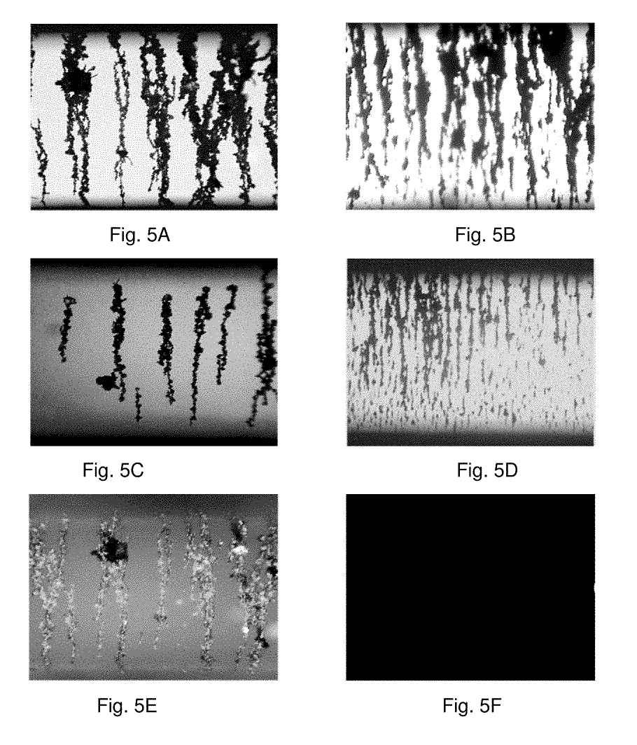

FIG. 5 is the comparative experiment results of the present invention and the existing on-line visual ferrograph. Six ferrograms were obtained from the gearbox oil, gasoline engine and diesel engine oil with the oil transmittance of 80%, 9.5% and 0.5%. It is clear that the image resolution of ferrograms of the present invention is not affected by the transmittance of the oil. The contrast between the ferrograms and the background is obviously. When the transmittance of the diesel engine oil is only 0.5%, the contour feature of wear debris of present invention is still clear (as shown in FIG. 5E). On the contrary, the image resolution of wear debris of existing technology is susceptible to oil interference, and the existing technology is not applicable to acquire wear debris information of oil when the transmittance of the oil is only 0.5% (as shown in FIG. 5F).

1. A debris direct reflection embedded microscope including:
a focus mechanism configured to achieve different magnifications of wear debris;
a reflected light source;
a transmission light source as the background light source means for achieving quasi-uniform illumination and improve the image contrast of ferrograms;
wherein said focus mechanism is composed of a focus threaded sleeve, a limit threaded sleeve, a lens sleeve and a connecting seat, said focus mechanism and said reflected light source are placed in the internal space of a electromagnetic debris deposition device, and the center optical axis of said focus mechanism coincides with the center axis of the electromagnetic debris deposition device.

2. A debris direct reflection embedded microscope according to claim 1 wherein said focus mechanism and said reflected light source are fixed in said connecting seat, and said connecting seat connects said focus mechanism and said magnetic poles together.

3. A debris direct reflection embedded microscope according to claim 1 wherein said reflected light source is fixedly connected with the lower end of said lens sleeve.

4. A debris direct reflection embedded microscope according to claim 1 wherein the upper end of said lens sleeve has several slots, and the lower end of said limit threaded sleeve has the same amount of bosses;
said limit threaded sleeve is mounted in the slots by the bosses, and said focus mechanism is fixedly connected to said connecting seat by the screws on the both sides of said limit threaded sleeve.

5. A debris direct reflection embedded microscope according to claim 1 wherein said transmission light source is a bi-color flat light source composed of two monochromatic light A and B, a light source A is located at the center of the transmission light source, and a light source B is arranged at the edge of said A light source, and the wavelengths of said A light source and said B light source are different.

6. An adjustment method of the light intensity of transmission light source and reflected light source including:
detecting the background image when light intensity of the short wavelength light source is the maximum, and calculating the transmittance of the oil;
selecting the color of said transmission light source according to the transmittance of the oil;
adjusting the light intensity of said transmission light source and reflected light source to improve the image contrast of ferrograms.

7. An adjustment method of the light intensity of transmission light source and reflected light source according to claim 6 wherein said selecting the color of said transmission light source is operable by:
selecting short wavelength light source as the background light source when the transmittance of the oil is high, and switch off the long wavelength light source;
selecting long wavelength light source as the background light source, when the transmittance of the oil is low, and switch off the low wavelength light source.
